(19) United States
(12) Patent Application Publication
(10) Pub. No.: US 2022/0008087 A1
(43) Pub. Date: Jan. 13, 2022

(54) SURGICAL TOOL AND FIXATION DEVICES

(71) Applicant: MFr Technologies, Inc., Maple Grove, MN (US)

(72) Inventors: Trevor Jacob Laughlin, Minneapolis, MN (US); William D. Armstrong, Memphis, TN (US)

(21) Appl. No.: 17/369,687

(22) Filed: Jul. 7, 2021

Related U.S. Application Data
(60) Provisional application No. 63/048,933, filed on Jul. 7, 2020.

(51) Int. Cl. A61B 17/16

(52) U.S. Cl. CPC A61B 17/1604; A61B 2017/00367

(57) ABSTRACT

The present invention comprises a device and system for delivering implantable bodies for anchoring human tissue and bony anatomy. The system may comprise a housing with a handle, an advancement mechanism, a hollow shaft, and a plurality of implant bodies. The present invention also describes a fixation device, such as a dart, staple, screw, or rivet so that the housing includes a handle comprising a lever or trigger for advancing implants, and optionally comprising a second lever or trigger for a second operation. The surgical device may also include an impacting mechanism and a manual advancing mechanism for advancing an elongated body into and through bone, with an optionally reverse setting which changes the direction to retract the elongated body. The invention further includes methods for using the surgical device.

### CROSS-REFERENCE TO RELATED APPLICATION

The present application claims the benefit of U.S. Provisional Application No. 63/048,933 entitled “SURGICAL TOOL AND FIXATION DEVICES”, filed Jul. 7, 2020, which is incorporated herein by reference in its entirety.

### TECHNICAL FIELD

The present invention is generally directed to a device and method for surgery, and more particularly for delivering implantable bodies for anchoring human tissue and bony anatomy.

### BACKGROUND OF THE INVENTION

Surgical procedures require precision and control. There are a number of procedures dealing with orthopedic surgeries that require such precision and control. One such example is an osteotomy, or the surgical cutting of bone. A pedicle subtraction osteotomy is a surgical procedure to correct certain deformities of the spine. A spine with too much or too little curvature can be corrected. During a pedicle subtraction osteotomy, areas of bone are removed. The spine is then realigned and stabilized in its new alignment.

Fixation points are placed with pedicle screws. Curettes of various shapes and sizes, and a high speed burr is typically used to decancellate the vertebral body. Pedicle osteotomes are used to cut and remove the pedicle. Rods are placed, and the posterior wall is impacted into the vertebral body. Rods are adjusted, the spine is moved, and rods are adjusted again until stabilization is achieved.

Another such example is a kyphoplasty, a vertebral augmentation surgery to treat fractures in vertebrae from osteoporosis or trauma. This procedure involves injecting acrylic bone cement or filler material into the fracture site. In doing so a surgeon can stabilize the vertebra, restore it back to its normal height, and consequently reduce pain from the fracture.

In oblique lateral interbody fusion, or OLIF procedures, a neurosurgeon accesses and repairs the lower (lumbar) spine from the front and side of the body (passing in a trajectory about halfway between the middle of the stomach and the side of the body). This is a less invasive approach to spinal fusion surgery involving the removal of damaged intervertebral disc and bone, and fusing two adjacent spinal vertebrae. In doing so, a surgeon can minimize cutting to muscles and uses a single port to access the disc space, fill it with bone material and then fuse the bones of the lumbar spine. Fusion can utilize bone graft material taken from the patient (autograft), a cadaver (allograft), or a synthetic substitute.

In certain circumstances, spine surgeons perform disc repairs or discectomies. Discectomy is a common surgery for treating herniated discs in the lumbar region. In this procedure, the portion of the disc that is causing pressure on a patient's nerve root is removed. In some cases, the entire disc is removed. Nerve root damage can occur as a complication. Microdiscectomies under special microscopes with relatively smaller incisions are routinely performed in order to minimize damage to surrounding tissue. In some cases, a laminotomy or laminectomy is first needed to provide access and visibility to the disc.

Laminectomy is surgery that creates space by removing the lamina, the back part of a vertebra that covers a patient's spinal canal. Also known as decompression surgery, laminectomy enlarges the spinal canal to relieve pressure on the spinal cord or nerves.

This pressure is most commonly caused by bony overgrowths within the spinal canal, which can occur in people who have arthritis in their spines. These overgrowths are sometimes referred to as bone spurs, but they're a normal side effect of the aging process in some people. Complications can arise when injury to the spinal cord's dura or nerve roots is incurred during surgery.

In endplate preparation, cartilage must be thoroughly removed for good attachment to bone morphogenetic proteins (BMP's). Controlled separation of the cartilage from the cortical bone endplate in the disk space is performed. Careful application of force parallel to the cortical endplate beneath the cartilage surface is made in preparation for fusion and Interbody placement. Too much force damages the cortical bone leading to subsidence and too little force does not clear the cartilage off of the bone, and affects fusion potential. It is difficult to do this in a simple reproducible manner. The current method is to strip all of the cartilage off of the cortical bone endplate without damaging the cortical endplate. It is difficult to see all of the surface area to do this well and to prevent damage to endplate bone. In fusion surgeries, 4 screws are often used per disk level—sometimes as many as 5 levels.

Suture anchors, or ligament anchors are often used in orthopedic surgeries where ligament repair is performed. Anchors must be easy to insert, provide a firm anchor in bone, simple, reliable, and strong. Anchor site surfaces are oftentimes rounded and slippery, and present very hard cortical bone. Anchors must firmly affix to the underside of the cortical layer without causing delamination of the cortical layer from the underlying bone. Anchors are typically bio-absorbable or metallic.

Fixation implant delivery is a common requirement in orthopedic procedures. Placing various types of anchors in and around bony anatomy for orthopedic surgery is a lengthy and highly manual process. Precise placement of the holes, screws, and anchors can be challenging as target surfaces are slippery and/or movable. Thus, there are a number of surgical procedures that require the use of a device that allows for great precision and control.

### SUMMARY OF THE INVENTION

The present invention comprises a device and system for delivering implantable bodies for anchoring human tissue and bony anatomy. In its primary embodiment, the system comprises a housing with a handle, an advancement mechanism, a hollow shaft, and a plurality of implant bodies.

For example, one embodiment of the present invention employs a method of first placing bone anchors into the spinous process using ligaments to compress for increased lordosis; then tying down spinous processes; then inserting an anchor into each one; and then tightening down the ligament. Ligaments may be attached to wire or to the end of rod constructs to provide fixation. Rapidly deployable anchors are made possible with a mechanized delivery tool. Such an apparatus can be used for fusing ligaments together, as in an extension of laminar fusion. In an alternative embodiment the aforementioned ligament could be exchanged with wire instead. Going quickly, one shot after another, is beneficial especially in short hyperangulated plates where, for example, 4 screws are affixed per vertebral body.

One embodiment of the present anchor implant delivery invention would be akin to a nail gun with a cartridge. In a disc repair scenario, the apparatus disclosed herein could be used as follows: Surgeon cuts the annulus, removes herniation, and then sutures the annulus flap. The surgeon could then use the device to pin the annulus back together rather than using a conventional suture. The automated assistance of the present invention adds the additional possibility and utility of delivering an anchor off-axis of a primary shaft portion—for example delivering a rivet at an angle into a lumbar implant. Such utility is similarly beneficial in the posterior cervical fusion or in posterior plates. A cortical trajectory is introduced, enabling a user to insert implants in a controlled fashion rather than archaic manual impacting. The internal mechanism of the device may result in a distal push and/or a proximal pull of the tip and anchor. Additionally, the shaft is optionally bayoneted and used through a slotted tube off axis for visualization of the tip.

In one embodiment, each anchor delivery is achieved with an automated assist which can deliver sufficient velocity and force to overcome and penetrate dense, hard cortical bone. For example, and not a limitation, automated assist may comprise a spring, compressed fluid, electromechanical conversion, other energy storage means, or some combination thereof.

In one embodiment, each anchor delivery from the device described herein allows for more than one force application. For example, an impacting mechanism may be actuatable to initiate pilot hole with distal anchor surface or with a separate pilot hole maker, and a subsequent advancement may be affected with the same or a secondary mechanism. Alternatively, post-pilot hole advancement may be achieved utilizing the first mechanism at a lower force application.

The present invention describes both a delivery tool, and a fixation device, such as a dart, staple, screw, or rivet. One example of an apparatus and system for fixation implant delivery includes a housing with handle comprising a lever or trigger for advancing implants, and optionally comprising a second lever or trigger for a second operation. By way of example and not a limitation, a first lever or trigger may create a pilot hole, and a second lever or trigger may advance or insert an implant into the surgical site. An advancement mechanism may be included, optionally configured for a single-stage insertion whereby actuation drives the implant into the surgical site to its full and final depth.

Optionally configured is a dual-stage insertion whereby a first stage creates a pilot hole and a second stage advances and inserts an implant. By way of example and not a limitation, pilot hole creation may be actuated by applying pressure at the tip, and the implant advancement may be actuated by squeezing a lever. Further, optionally included is an indexing mechanism for controlling depth and delivery. Optionally included is a safety mechanism which is disarmed by a user-controlled switch, or by applying pressure at the tip. A hollow shaft is optionally “bayoneted” wherein the distal axis portion is offset from the proximal axis portion. A tip is optionally specially configured to release implant bodies individually. Optionally included is an internal profile variation such as a ledge, a detent, a slot, a taper, a spiral, or other change from continuous smooth barrel for the purpose of activating an anti-backout mechanism of implants. A plurality of implant bodies is optionally included in or coupled to the housing, each implant including an anti-backout mechanism or feature. Optionally included in each implant body is one or more reverse direction barb. Optionally, threads are included near the proximal head for cortical layer fixation and as a fail-safe for a user to ensure full depth penetration. Implants are optionally configured to expand radially once inserted into a surgical site and after leaving a hollow shaft with the inclusion of tip slot(s) or open geometry for material spring force flexure. A hinged locking insert is optionally included to create an automatic snap lock upon leaving the shaft tip. In one embodiment, torsion at the head causes expansion of the embedded portion. With threads included, the implant may simultaneously advance into the bone, optionally compress axially, and expand barb or barbs radially.

Implant anchors may be in the form of expanding pop rivets, which may be loaded into handle housing via a cartridge. Anchors may be removably connected, one after another, or nested such that the distal-most portion of one body is positioned distally from the proximal-most plane of the next distal-most body. Each anchor is optionally cannulated for guidance and placement, for example using robotics or surgical navigation systems. Expansion may take place mechanically, or alternatively using fluid pressure or a chemical reaction, or some combination thereof.

Fixation implants may be used as intrinsic fixation for interbodies, for example in lateral mass plates, laminoplasty plates, or anterior cervical plates. Anchor or screw head optionally interfaces with a plate, and flanges (rivets) fan out as “anti-backout” implements. In one embodiment, insertion and locking takes place during the same step or stroke. For example and not limitation, the implant may be around 3.5 mm in diameter, as this size is known to work well for delivery into relatively dense bone.

Anti-backout barbs extend outward radially from the primary hole axis. The barbs may comprise implant-grade metal such as titanium, nitinol, or stainless steel. Alternatively, the barbs may comprise bio-absorbable or medical polymers. In one embodiment, the implant comprises both metal and polymer features, such that efficient force transmission is achievable through a relatively high stiffness metal core, and force on the underside of the cortical bone layer can be managed through polymer barb features with relatively lower stiffness and hardness. Alternatively, stiffness can be managed utilizing the same material with a different cross-sectional profile, such as a multitude of thin metal wires which can contract to form barbs. In yet another alternative embodiment, a ceramic, artificial graft material, or flowable substrate is utilized for an anchor. By way of example and not limitation, ceramic, artificial graft material, or flowable substrate may be used in conjunction with metal or polymer frame, and act as a malleable or crushable backfill, distributing even pressure on the cortical underside when the implant is set.

In orthopedic surgeries, bony substrates sometimes need to be added to the surgical sites, such as in kyphoplasties with expandable devices. Current methods of filling these sites are highly time-consuming, inefficient, and taxing. Substrates are forced into a funnel through a cannula with a push rod, which often gets stuck and needs to be re-worked. The present invention discloses a handheld surgical device and system which enables flowable bone graft or other filler material into surgical sites. The device housing includes a mechanism which advances filler material through a hollow shaft, at the user's demand, such as a caulking gun. Filler material may be introduced as discrete pieces of bone grafts or as a slurry, a gel, allograft, autograft, ceramics, demineralized bone matrices, demineralized bone fibers, or other ductile material. Cartridges may be packaged in pre-loaded sets for loading into the device housing, optionally with different cartridges including different materials. Alternatively, the device could include an opening for inserting a material of the surgeon's choice, such as allografts, autografts, bone cement, ceramics, demineralized bone matrices (DBM), demineralized bone fibers (DBF), or others. Alternatively, a device may come with a predetermined amount and type of material. Bone graft/biologics delivery tool to disk space, to interbody devices after implanted for retro fill such as in expandable devices. Guidewires are often used in orthopedic surgeries to guide and precisely place cannulated screws. Pilot holes and guide channels are often created to increase the accuracy and precision of subsequent operations. Biopsies are often performed on bone and bone marrow for pathologic analysis.

The present invention discloses a handheld surgical device which impacts and advances an elongated body into and through bone. The primary device embodiment includes an impacting mechanism and a manual advancing mechanism, and optionally a reverse setting which changes the direction to retract the elongated cutting body. The impacting mechanism is essential in initializing the delivery into and through hard bone. Relatively high velocity impacts result in less chance of buckling in very fine or small diameter cutting wires. After the tip of the elongated body is through the cortical layer, the manual advancement mechanism allows the operator to move the tip forward at a known distance with precise control. Manual advancement is optionally performed by means of squeezing a lever akin to a caulking gun, a familiar and easy to use operation. Once a sufficient depth of penetration is reached, an elongated cutting body, optionally a guidewire, can be released, or retracted. In an alternative embodiment, the biopsy sample can be removed from the surgical site for transfer to a laboratory.

In another alternative embodiment the device drives a cutting implement proximally and may also rotate it at the same time, such as an impacting drill. Such a manual drill gives the user a heightened ability for tactile feedback as the cutting implement is advanced, which is highly useful for detecting changes in bone density. Skiving, or slipping of a cutting instrument, is commonplace in robotic assisted surgical operations as well as fully manual surgical operations. It is not always apparent when skiving occurs, until after the hole is created and the collateral damage is already done. Precise hole creation achieved by the present invention greatly reduces the chance of skiving and associated collateral damage. The automated creation of these pilot holes is additionally beneficial in percutaneous applications where visibility is limited. In yet another embodiment, an implant could be attached to the end of the tool, and the advancement mechanism could be used to implant interbodies. Another application of the present invention is craniofacial surgery, where small precise holes are often needed. Yet another application is for bi-cortical pilot hole generation, which requires careful advancement and depth management.

Rapid acceleration of a weighted transfer carriage may be utilized to impact or thrust the cutting element toward the surgical site of interest. This acceleration results in an impulse which may cause noticeable push-back or recoil if the placement of the tip is resultantly moved. Movement of the tip upon or immediately before impact may be mitigated by suspending the relative position of the tip guide with respect to the primary housing such that the housing can move away from the surgical site and the distal tip guide can remain on the surgical site surface. Alternatively, the elongated cutting body may be loosely coupled to the energy transfer housing. In each of the two aforementioned cases, a spring, damper, or buffering medium can be utilized to create necessary compliance to decouple the energy transfer housing and the cutting tip guide, thereby enabling constant pressure at the distal tip guide surgical surface interface.

Described herein is an anatomic tissue cutter, penetrator or mechanical osteotome. In osteotomies, a surgeon typically is viewing a surgical site with limited visibility while swinging a mallet into a cutting tool. The surgeon must be careful not to swing the mallet too hard or two softly, and not to let the tip skive or slip out of place. Further, if there is sensitive anatomy such as a nerve nearby, the surgeon likely must focus on that nerve so as to avoid hitting and damaging it. As a result, the surgeon is not able to simultaneously view the mallet or receiving surface of the cutting tool and provide the attention necessary to control force delivery. For this reason an automated mechanism for delivering a known force or achieving a known penetration distance is beneficial in adding a level of control for the surgeon. Capillary flow of small holes and precise creation of a bony defect without affecting the integrity of the global bony cortical endplate reveals an intriguing combination of endplate preparation for bone fusion. The totality of the cartilage may be accomplished by the combination of capillary flow through the bone and up through the cartilage while making controlled defects in the cortical surface that does not result in weakening and subsequent subsidence and free the disc, but keep the cartilage. The instrument can penetrate the cartilage and endplate allowing for fusion without cartilage removal. This may be supplemented with BMP. This creates the potential of purposeful pseudoarthrosis. Getting the biggest part of the disk out of the way is easy, one must remove all of the cartilage to get a good fusion with BMP; for example in an endplate preparation. Using an angled guillotine cutting implement attached to the mechanized impactor may additionally be beneficial in a laminectomy. A safety guard may be used in conjunction with the primary device, while controlled mechanical impaction under fluoroscopy is safely performed. In an alternative embodiment, an osteotome delivers a controlled force off the primary axis while using a surgical robot. The controlled impact or force delivery of the present invention gives the user more control and makes the cutting tip less likely to problematically follow paths of least resistance, where said paths lead to sensitive anatomy. Each charge of the device may result in one or more impacts, depending on the size of the bone or the operation. Another decortication of facets, for example in posterior lumbar interbody fusion (PLIF) procedures. Interchangeable tips may be included for cutting various surfaces and/or effecting different motions such as pushing, pulling, vibrating, oscillating, or reciprocating.

In one embodiment, a base device is provided which converts energy of one type into mechanical, kinetic energy. Kinetic energy can then be used to create cutting movement. In one embodiment, base device includes an energy storage element which can be charged by the user. The energy storage element may produce rotation of a shaft with a coupling on the end, on which various shafts and tips can be affixed. Each shaft or tip may produce the same or different motion via its own transmission means.

Cartilage removal is necessary in many orthopedic surgeries, such as lateral spinal surgeries where fusion and interbody fixation are augmented. In these procedures, much cartilage must be removed from the cortical bone endplate. Too much force damages the cortical bone leading to subsidence and too little force does not clear the cartilage off of the bone and affects fusion potential.

The present invention discloses a handheld surgical device and system for removing cartilage in orthopedic surgeries. The device comprises a mechanical actuation and a moveable tip configured to scrape cartilage without substantially damaging cortical endplates. Optionally, the system includes exchangeable tips with different configurations for specific cartilage removal scenarios. The device described herein presents a means to clear the cartilage quickly without damage to the cortical endplate. In one embodiment, a cartilage removal tool is introduced with a mechanical reload for rapid fire and reciprocation or vibration, akin to a jackhammer or concrete breaker. The device may be mechanically assisted with an internal energy storage element such as a spring or a battery, or an external power source. A scraping device may be introduced that acts like a curette for cutting during pushing, pulling or both. Sometimes it is beneficial to only pull in order to safely avoid the spinal cord. Different tips are optionally included, such as in typical disc prep kits. Different tips could be included for different anatomy or for different procedures. A controlled, mechanically-assisted cartilage removal method is valuable in removing enough, but not too much tissue, while visibility and leverage are limited, such as in minimally invasive lateral access spine surgeries. Another application is removal of angular cartilage for cortical bone penetration without removal of the cartilage layer.

Any of the aforementioned inventions may be combined in a specialized system or surgical procedure. In one embodiment, devices are used to break up the facet in a predictable way and then backfill with fusion material and attach a bone anchor. Ideal perforations can be created to facilitate bone fusion. Whereas driving rods percutaneously there is poor access to laminar facet joints, applying the devices of the present invention percutaneously may be used to affix crushing together. For example, a user could create a hole and implant something that would backfill such as a graft.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A depicts an implant delivery apparatus.

FIG. 1B depicts a pressure-limiting safety lock for a surgical device.

FIG. 2A depicts a combination charging and triggering system in a first position.

FIG. 2B depicts a combination charging and triggering system in a second position.

FIG. 3A depicts a plurality of removably attached fixation implants in a first embodiment.

FIG. 3B depicts a plurality of removably attached fixation implants in a second embodiment.

FIG. 4 depicts a delivery mechanism for a plurality of fixation implants.

FIG. 5 depicts a nested arrangement of fixation implants.

FIG. 6A depicts a locking bi-stable anchoring implant and a spring-assisted anchoring implant delivery.

FIG. 6B depicts a spring-assisted anchoring implant delivery FIG. 7A depicts a nested implant inserter upon delivery.

FIG. 7B depicts a nested implant inserter upon retraction.

FIG. 8A depicts an implant insertion system comprising a pilot hole creator and a pusher.

FIG. 8B depicts an implant insertion system creating a pilot hole.

FIG. 8C depicts an implant insertion system delivering an implant.

FIG. 9 depicts a tissue anchor with an internal channel or cannula.

FIG. 10 depicts a tissue anchor with an internal void for radial or transverse flexibility.

FIG. 11 depicts a tissue anchor with multiple voids for flexibility.

FIG. 12A depicts a locking bi-stable anchoring implant in a first position.

FIG. 12B depicts a locking bi-stable anchoring implant in a second position.

FIG. 13A depicts a tissue anchor with an expandable tip in an initial position.

FIG. 13B depicts a tissue anchor with an expandable tip in a final position.

FIG. 14 depicts a mechanically assisted apparatus for advancing grafts or filler through a cannula.

FIG. 15 depicts a second embodiment of a mechanically assisted apparatus for advancing grafts or filler through a cannula.

FIG. 16A depicts a first tip for cutting tissue.

FIG. 16B depicts a second tip for cutting tissue.

FIG. 17 depicts a force-aligned handle configuration for a mechanically assisted tissue removal surgical device.

FIG. 18A depicts a first motion profile for a reciprocating tissue removal device embodiment.

FIG. 18B depicts a second motion profile for a reciprocating tissue removal device embodiment.

FIG. 18C depicts a third motion profile for a reciprocating tissue removal device embodiment.

FIG. 19 depicts a modular surgical apparatus and system with removable and exchangeable parts.

FIG. 20A depicts a sharp scraping tip comprising a bent flat wire from the front.

FIG. 20B depicts a sharp scraping tip comprising a bent flat wire from the side.

FIG. 21A depicts a first tip configuration for tissue removal.

FIG. 21B depicts a second tip configuration for tissue removal.

FIG. 21C depicts a third tip configuration for tissue removal.

FIG. 21D depicts a fourth tip configuration for tissue removal.

FIG. 21E depicts a fifth tip configuration for tissue removal.

FIG. 21F depicts a sixth tip configuration for tissue removal.

FIG. 21G depicts a seventh tip configuration for tissue removal.

FIG. 21H depicts an eighth tip configuration for tissue removal.

FIG. 21I depicts a ninth tip configuration for tissue removal.

FIG. 22A depicts a front view of a surgical cutting tip with a tapered end.

FIG. 22B depicts a front view of a surgical cutting tip with a tapered end and including a second cutting portion.

FIG. 22C depicts a side view of a composite cutting tip.

FIG. 23 depicts a surgical tool with user adjustable tip extension

FIG. 24 depicts a handheld mechanically assisted impacting surgical tool

FIG. 25A depicts a first actuator handle configuration which may enable two or more mechanisms.

FIG. 25B depicts a second actuator handle configuration which may enable two or more mechanisms.

FIG. 25C depicts a third actuator handle configuration which may enable two or more mechanisms.

FIG. 25D depicts a fourth actuator handle configuration which may enable two or more mechanisms.

FIG. 26A depicts an indexing impact mechanism in a first position.

FIG. 26B depicts an indexing impact mechanism in a second position.

FIG. 26C depicts an indexing impact mechanism in a third position.

FIG. 26D depicts an indexing system component which enables a retraction function

FIG. 27 depicts a surgical tool with three actuators

FIG. 28 depicts a handheld mechanically assisted osteotome in use

FIG. 29 depicts a surgical tool deploying a sharp tip to a known distance

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

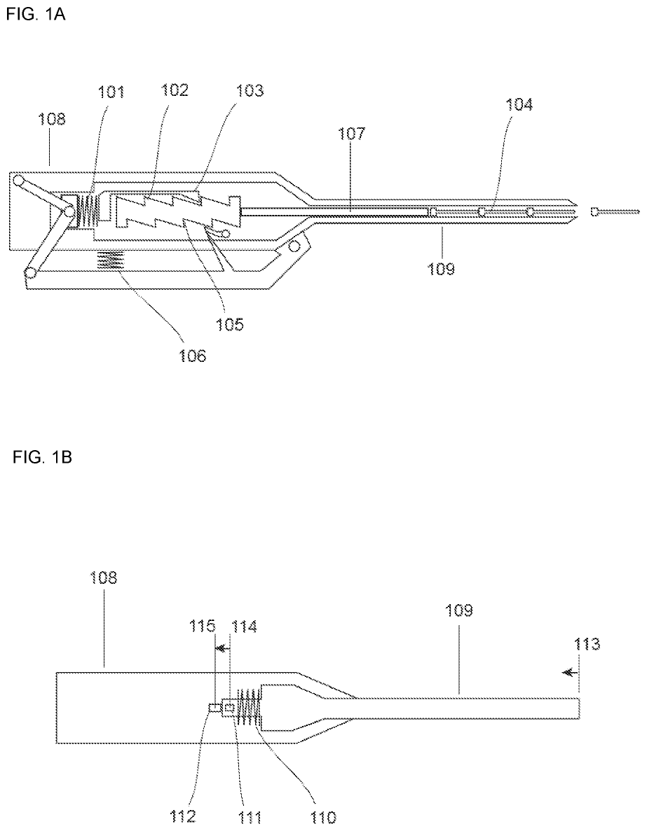

The implant delivery apparatus of FIGS. 1A and 1B comprises a handle portion 108 and a hollow shaft portion 109. Within the handle is an advancement mechanism including an elongated implant pusher 107 and a stepped index body 102, optionally with a second index 105. When the actuator 106 with optional biasing element is activated by the user, a push mechanism with optional spring 101 and index pusher 103 advances the index body distally until implant 104 reaches its final position with its proximal head at or near the distal tip of the hollow shaft. A minimum pressure limiting safety lock is optionally integrated whereby the hollow shaft 109 is movable with respect to the handle portion 108. In this embodiment, actuation is only possible when pressure is applied at the distal shaft tip 113, compressing a biasing element 110 proximally until safety lock initial position 114 reaches final position 115 and at which point alignment of shaft lock portion 111 and/or handle lock portion 112 provide an open path for actuation.

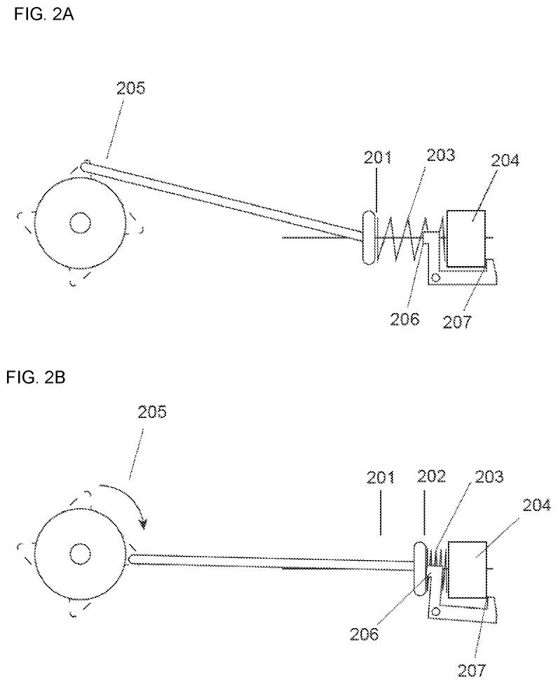

The combination charging and triggering system of FIGS. 2A and 2B shows a mechanism 205, optionally a slider crank, pushing a distal surface from a first position 201 to a second position 202, and in doing so charging a biasing element 203. At or near the end of the stroke, the distal pushing surface contacts a first portion of a movable latch 206, which in turn moves the second portion of the movable latch 207 and releases the impacting body 204 to freely advance via the biasing element.

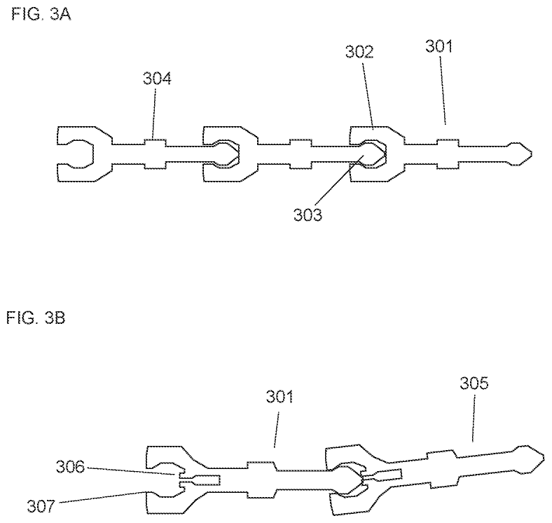

The removably attached fixation implants of FIGS. 3A and 3B comprise a first implantable anchor 301 and at least a second implantable anchor 305. Each anchor includes a proximal head 302 and distal tip 303, the distal tip of one anchor conjoining to the proximal head of the next anchor, distally, via coupling protrusions 307, magnetism, adhesive, mechanical connection or other means. Each anchor further includes one or more radial or transverse protrusions 304 for anti-backout function. Anchors optionally include one or more contact points 306, which, when pressure is applied, cause coupling protrusions to latch onto the tip of the next proximal most anchor. Conversely, when no forward pressure is applied, the distalmost anchor is free to separate.

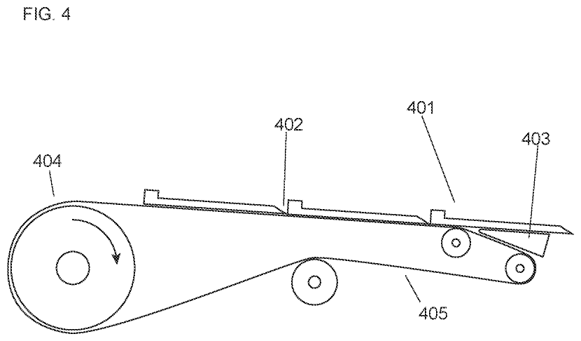

The fixation implant delivery mechanism of FIG. 4 depicts a closed loop conveyor 405 driven by a rotating mechanism 404 being utilized to deliver anchors 401 through a device and into a patient site. An intersecting body 403 guides the distalmost anchor out of the surgical instrument and into the patient site, and decouples it from the next distalmost anchor at the loosely connected separation point 402.

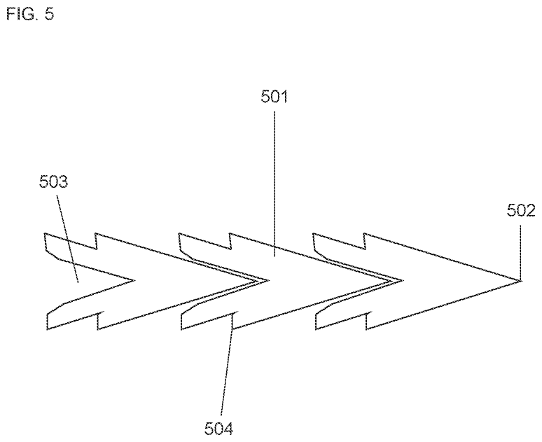

The nested arrangement of fixation implants in FIG. 5 illustrates three implantable fixation anchors 501, each comprising a tapered tip 502, a nesting void 503 and at least one transverse protrusion 504. The distal tip of one anchor fits into the proximal void of the next, distally.

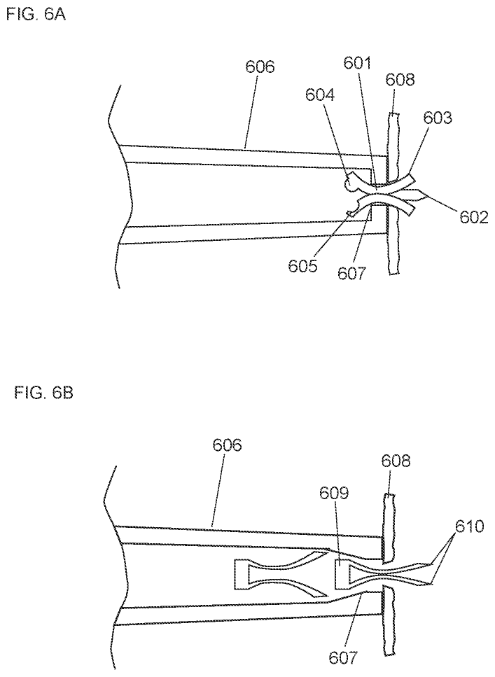

A locking bi-stable anchoring implant 601 and a spring-assisted anchoring implant 609 are shown in FIGS. 6A and 6B, respectively. The implants are delivered through a hollow shaft 606 with a distal tip 607 that is optionally narrower than the proximal tube portion to guide movable parts of said implants. In a first embodiment, a narrowed tube section guides a sharp tip 602 and one or more distal barbs 603 past the distal shaft tip and through the patient tissue 608. Upon being advanced further through said tip, a proximal portion with a first mating feature 604 and a second mating feature 605 is forced together until said first mating feature and said second mating feature meet, and are locked together, thereby locking distal barbs in a radially expanded configuration. In a second embodiment, two or more sharp tips 610 are forced apart via material spring force after passing through the distal shaft tip.

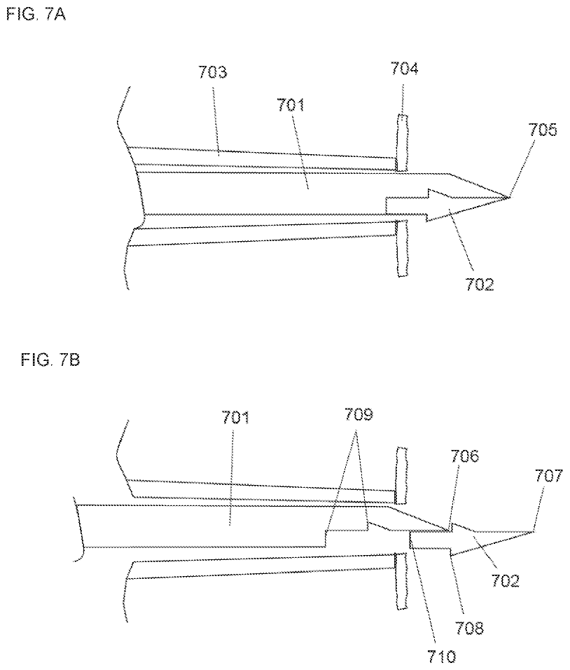

The nested implant inserter shown in FIGS. 7A and 7B depicts an elongate inserter 701 with a tapered tip 706 and including a cutout with at least one shelf 709 parallel or nearly parallel with patient site surface 704. A mating anchor piece 702 comprises a tapered tip 707 and at least one proximal face 710 for receiving applied axial force from said inserter. Tapered tip of the inserter and of the anchor piece enter the patient site together, through a hollow shaft 703, with a first combined tip configuration 705. Upon retracting the inserter, at least one transverse protrusion 708 grips the patient tissue, thereby maintaining implanted position.

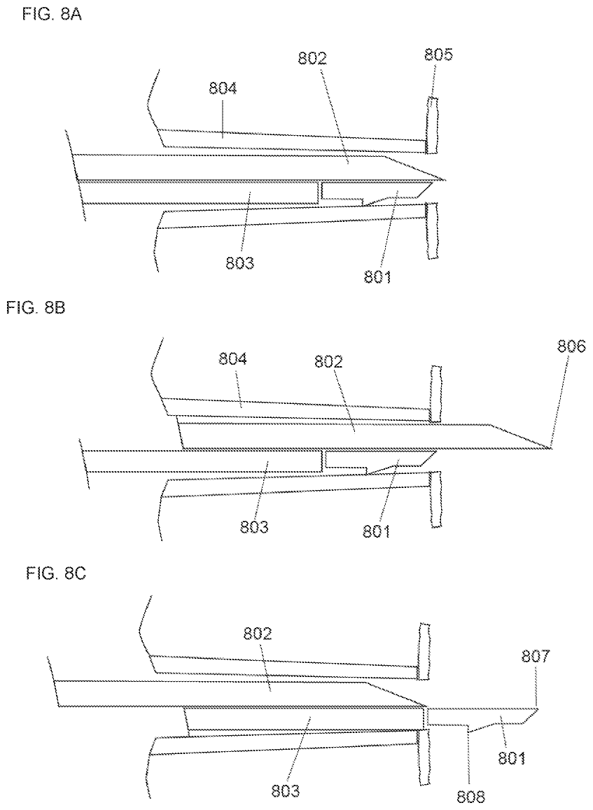

The implant insertion system of FIG. 8A through 8C depicts a pilot hole creator 802 with a tapered tip 806 and an elongate pusher 803. Pilot hole creator is first driven through a hollow shaft 804 to create an opening in tissue surface 805, and then retracted back into the shaft. Then, the elongate pusher is advanced distally, thereby inserting implantable anchor 801 into the patient site. Anchor includes a tapered distal tip 807 and at least one transverse protrusion 808 for gripping the tissue and maintaining implanted position.

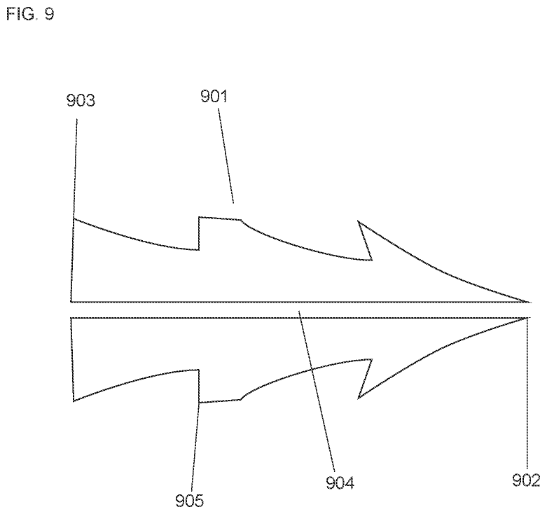

The tissue anchor 901 depicted in FIG. 9 comprises an internal channel or cannula 904 for robotic guidance and/or fluid flow, which may be passive, such as bone marrow from a medullary cavity. Anchor includes a proximal head 903, a tapered distal tip 902, and optionally one or more transverse protrusions 905.

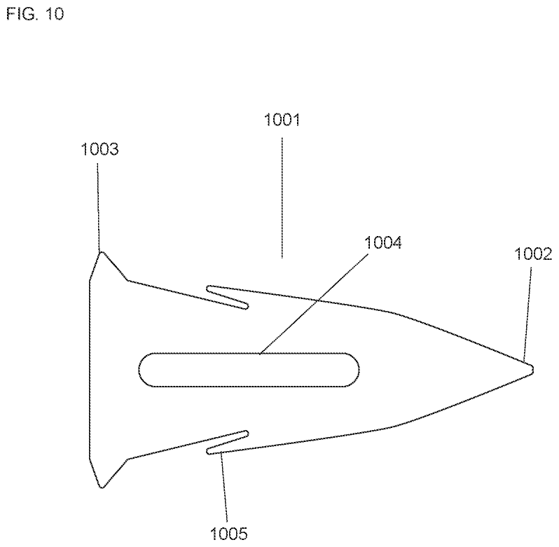

The tissue anchor 1001 shown in FIG. 10 depicts an internal void 1004 for producing radial or transverse flexibility and forcing one or more barbs 1005 outward after insertion into the patient site. The tissue anchor also includes a proximal head 1003 and a tapered distal tip 1002.

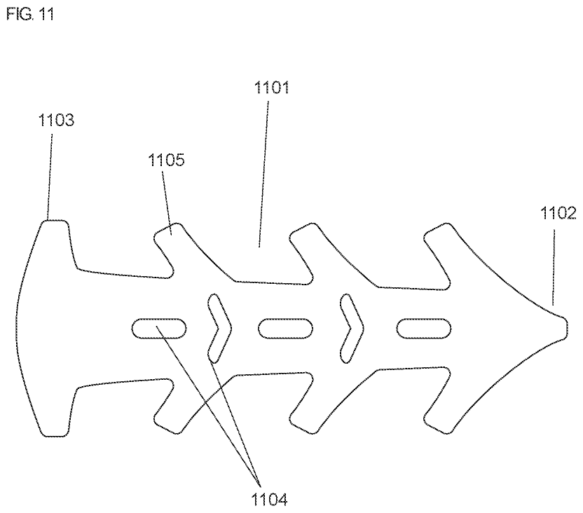

The tissue anchor 1101 of FIG. 11 includes multiple voids 1104 for flexibility. Specifically, these voids allow anti-backout barbs 1105 to narrow upon insertion, and widen after insertion is finished. A tapered tip 1102 is optionally included on the distal end and a wide head 1103 is optionally included on the proximal end.

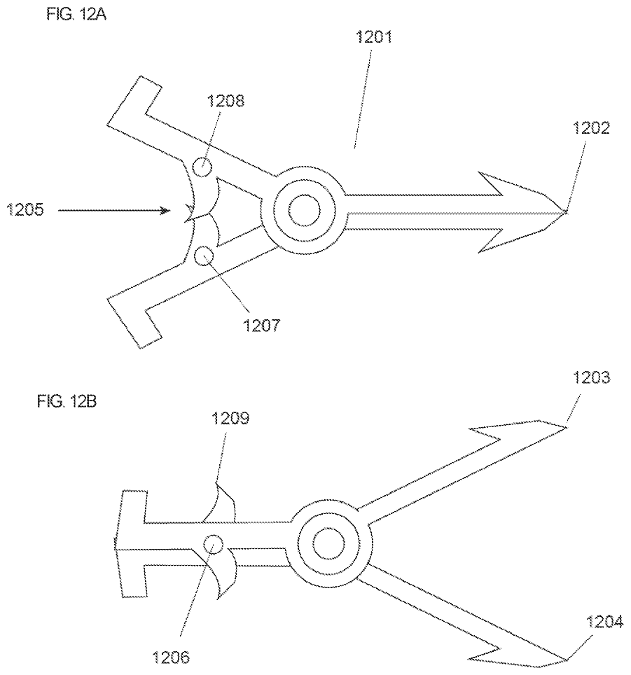

The implantable anchor of FIGS. 12A and 12B depicts a bi-stable apparatus 1201 comprising a first locking element 1208 and a second mating locking element 1207, which when mated, contain the apparatus in a second configuration. The apparatus includes a tip 1202 which splits into two tip portions 1203 1204 in its second configuration. Optionally included are barbs 1209 for digging into tissue and holding apparatus in place after insertion. Apparatus is activated into its second configuration when provided an applied force from a user 1205.

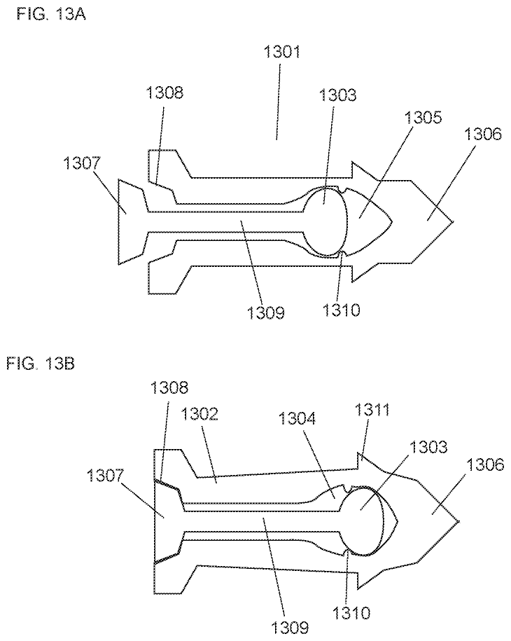

The tissue anchor 1301 of FIGS. 13A and 13B comprises an expandable tip 1306 with one or more radial or transverse protrusions 1311. Tip is expanded when a proximal actuation surface 1307 is activated, thereby forcing an expander 1303 from a first cavity 1304 into a second cavity 1305 via a connector 1309. One or more retainers 1310 are included to hold the expander in its final position within said cavity. A receiving cavity 1308 is optionally included to mate flush with said actuation surface. Outer cutting body 1302 optionally separate from the expander element.

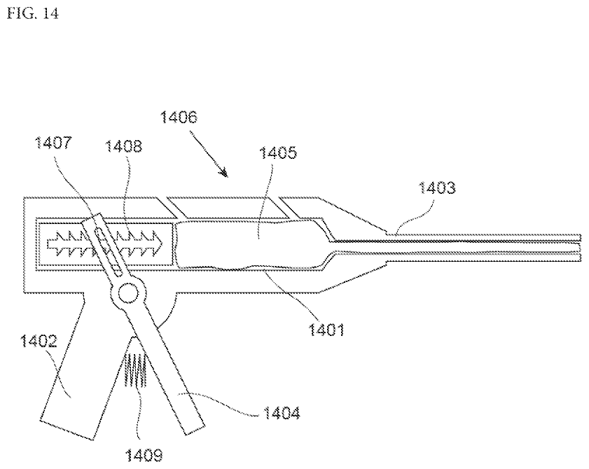

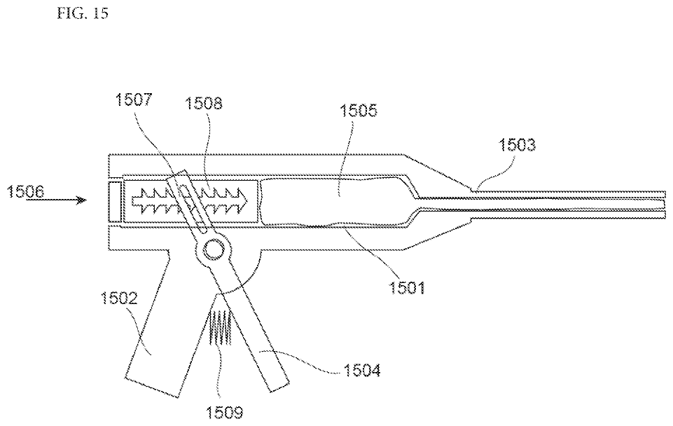

The mechanically assisted apparatus of FIG. 14 and FIG. 15 comprises a handle 1402/1502, a hollow shaft 1403/1503, an actuator 1404/1504, and a chamber 1401/1501. A biological graft or filler material 1405/1505 is advanced through the chamber and through a hollow shaft by means of a directional pusher 1408/1508 and its connection 1407/1507 to the actuator, the pusher being optionally indexed and unidirectional. The actuator is optionally returned to its home position after effecting actuation by a biasing element 1409/1509. Grafts or filler material is optionally inserted through an opening in the top 1406 or an opening in the proximal end 1506.

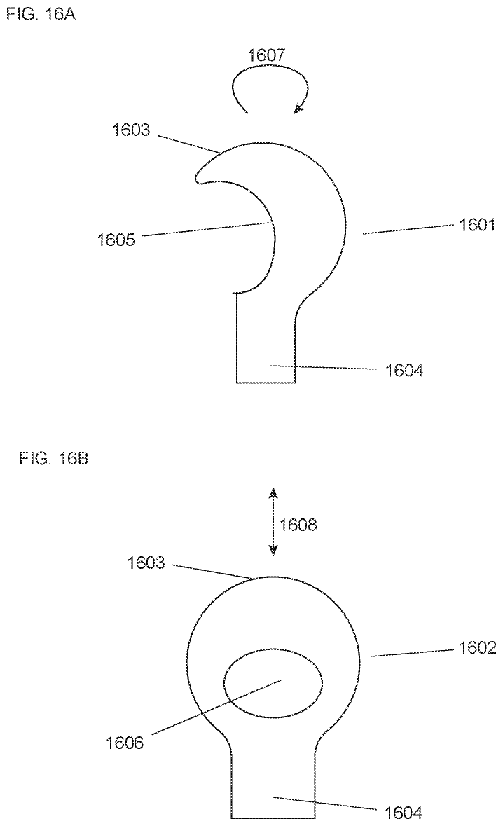

As shown in FIGS. 16A and 16B is a first cutting tip 1601 and a second cutting tip 1602, each configured for cutting or removing biologic tissue. Each aforementioned tip includes a sharp distal edge 1603 and a proximal end 1604. Proximal end is optionally connected to an elongate connector for limited access surgical procedures. Cutting tip optionally includes a side cutout 1605 or a center cutout 1606. The tip of the present invention is attached to a mechanism which drives the tip in an elliptical 1607 motion or a reciprocating 1608 motion.

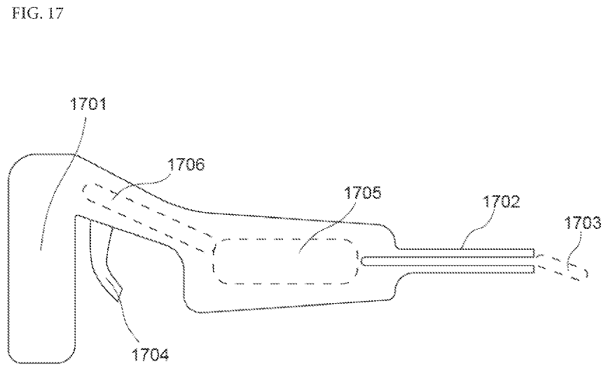

The surgical device of FIG. 17 comprises a handle 1701 configured in line or mostly in line with the axis of hollow shaft 1702 and/or the axis of cutting force. Cutting tip 1703 is optionally off axis from the hollow shaft. The tip is moved via an actuator 1704 connected to a drive mechanism 1705, optionally through a transmission mechanism 1706.

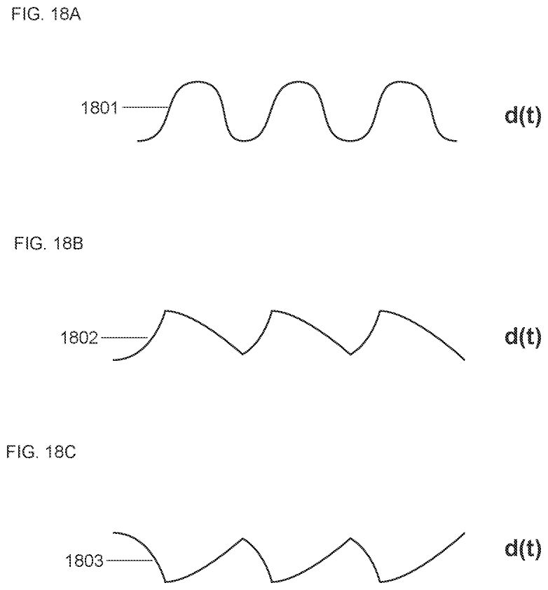

The cutting tip of the present invention may be configured to move in one of several motion paths for various applications, as shown in FIG. 18A through 18C. Motion may have a symmetric waveform period such as sinusoidal 1801, biased for higher pushing acceleration 1802, or biased for higher pulling acceleration 1803. In one embodiment of the present invention, a single device may be configured to move in any one of the aforementioned motions by way of modular switching or component exchanging.

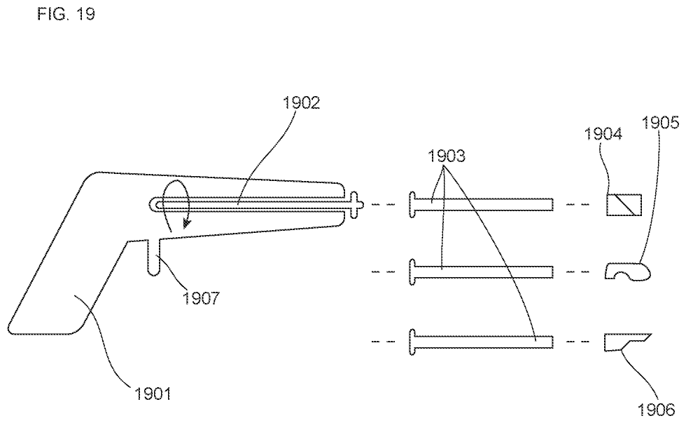

The modular surgical apparatus and system of FIG. 19 comprises a handle 1901, an actuator 1907, an internal drive mechanism 1902, one or more detachable shaft 1903, and one or more detachable tip 1904 1905 1906. Different shafts optionally include different geometries and optionally effect different tip motions via their respective transmission mechanisms. For example, specific tips may be configured for rotational cutting, impacting, or vibratory reciprocation.

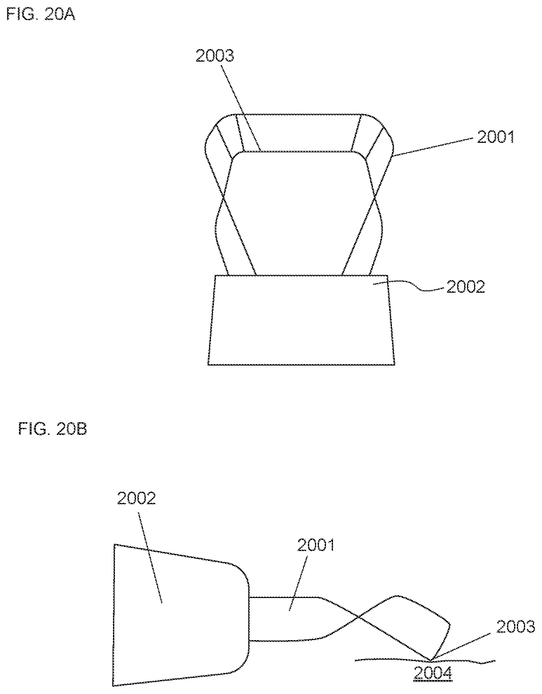

The surgical cutting tip of FIGS. 20A and 20B comprises a sharp scraping edge 2003 formed around a twisting and/or bent flat wire 2001 and protruding from a shaft end 2002. Sharp edge is formed for an alignment angled for cutting biologic tissue 2004.

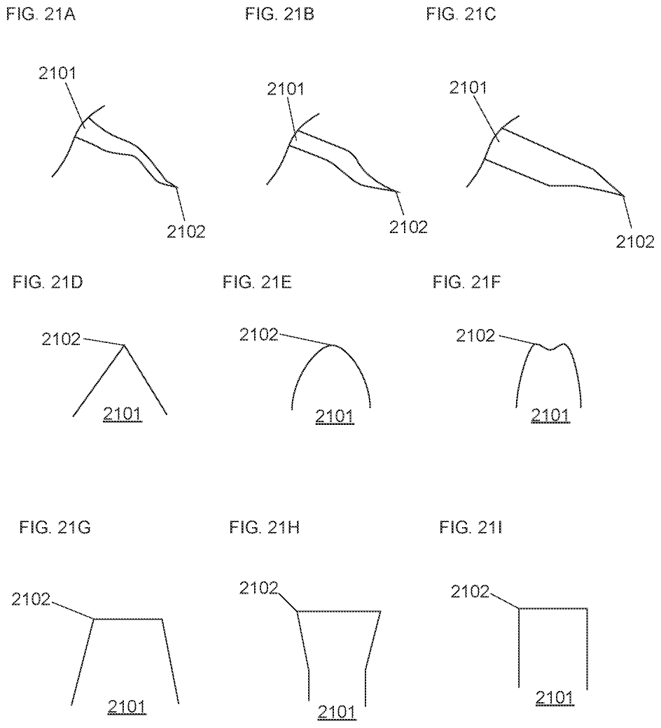

Multiple tip configurations for tissue removal are depicted in FIG. 21A through 21I. These tip configurations may be specially formed for applications of removing or cutting connective tissue or bone. Each tip has a cutting edge 2102 and a proximal portion 2101. Different tips are configured for different levels of flexibility and strength, and optionally for different motion profiles, for example smooth reciprocation versus impacting.

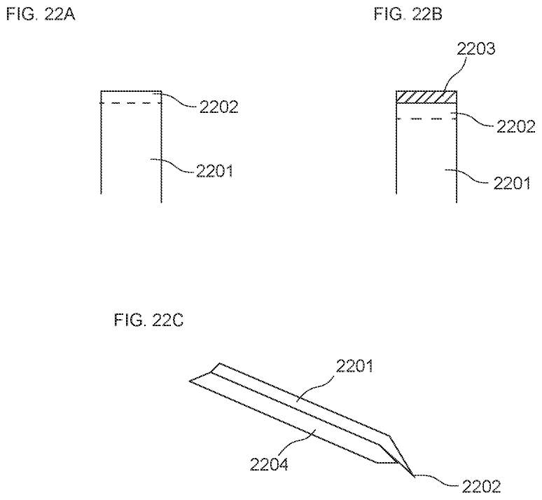

A surgical cutting tip may comprise a first material 2201 and at least a second material 2204, as shown in FIG. 22. The cutting tip comprises a tapered end 2202 and optionally a second tip portion 2203. The composite configuration of the present invention provides the user with greater control and tactile feedback when removing tissue by pushing, pulling, or scraping. For example and not limitation, a softer material 2204 may come into contact with bone or cartilage before or concurrently with a harder material 2201, thereby limiting the downward pressure of the cutting edge and the subsequent removed material thickness of a bone or connective tissue. One example use of this invention is the removal of cartilage on the surface of a bone, while limiting the damage done to the bone surface.

In one embodiment, a cutting tip comprises a soft backing with an array of cutting edges, akin to a rasp or sandpaper.

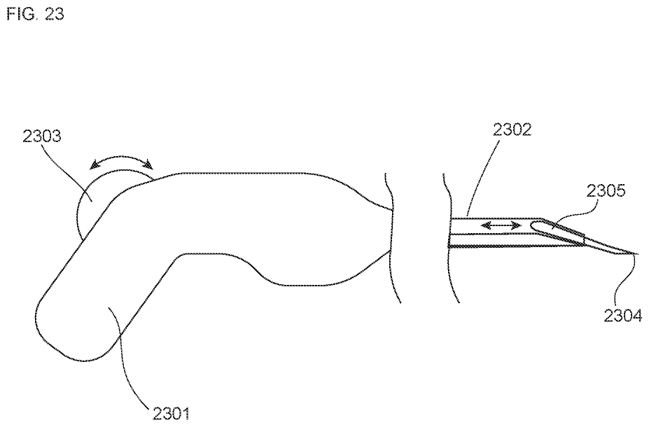

The surgical tool of FIG. 23 depicts a handheld device comprising a handle 2301, an adjuster 2303, a hollow shaft 2302, and a cutting implement 2305 with a sharp tip 2304. Upon manipulating the adjuster, the protrusion distance of the sharp tip with respect to the hollow shaft is increased or decreased, thereby controllably changing the depth of cut in a surgical operation.

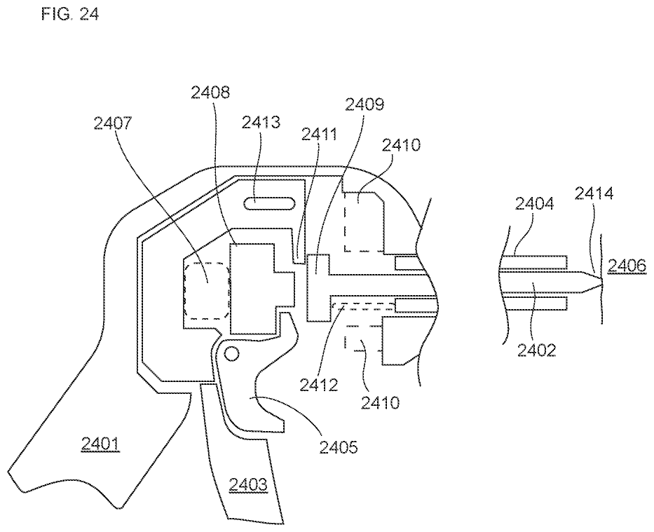

The handheld surgical tool of FIG. 24 comprises a handle 2401, a hollow shaft 2404, a cutting implement 2402 with a penetrating tip 2414, and an impacting mechanism 2407. The Impact mechanism is optionally driven by a spring. The surgical tool of the present invention comprises at least a first actuator 2403 and optionally a second actuator 2405 for effecting impacting. The cutting implement tip is pressed against the patient site tissue 2406 when a user applies pressure to the device handle. Optionally, a biasing element 2412 couples the cutting implement to the device body. The cutting implement is movable with respect to the hollow shaft. Upon activating one or more actuators, the impacting mechanism drives an impacting body 2408 toward a receiving portion 2409 of the cutting implement. Optionally, the impacting mechanism is charged a predetermined amount, providing the user with a predictable consistent force and/or penetration distance delivery. In an alternative embodiment, the user actively controls the charge level of the impacting mechanism via one or more actuators for various applications and impacts. A depth limiting stop 2410 may be included to limit the impacting body or cutting implement at a predetermined point. Said stop may comprise a soft material to act as a damper for reducing collateral damage of shaft momentum into patient tissue. Impacting mechanism may be movable with respect to the device body via a guiding track 2413 and a receiving portion 2411 which interfaces with the cutting implement. In making the impact mechanism movable with respect to the device body, a consistent impact force or travel distance can be achieved, even when the impacting element is pushed against patient tissue and into the device.

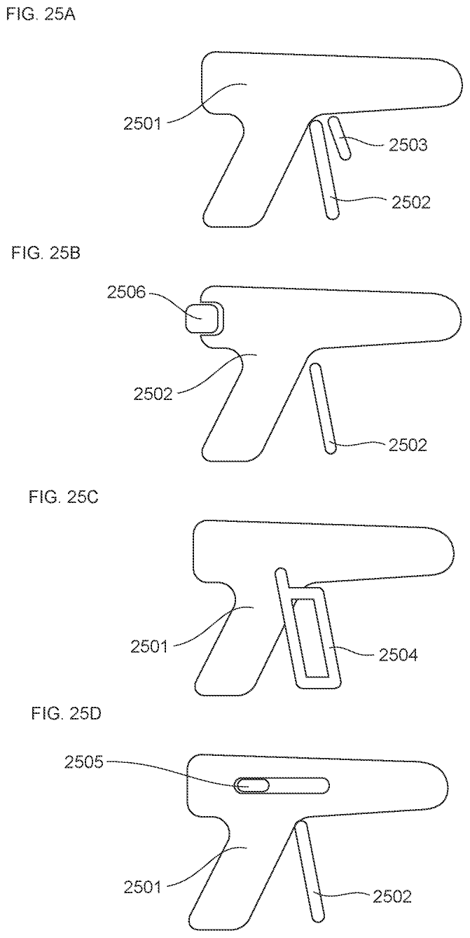

The handle configurations shown in FIG. 25A through 25D each comprise a handle 2501, and at least one actuator 2502 2503 2504 2505 2506. In a first embodiment, two actuators are included; one pull lever 2502 for charging, and one for impacting 2503. In a second embodiment, two actuators are included; one pull lever 2502 and one push button 2506. Said pull lever may be utilized for charging, and said push button may be used for impacting. Alternatively, pull lever may be utilized for advancing a cutting implement distally or retracting it proximally, depending on the position of said push button. In a third embodiment, a surgical tool comprises a single lever 2504 which can be both pushed distally or pulled proximally. In such a configuration pulling the lever may advance a cutting implement until the lever is pushed distally beyond a predetermined limit, at which point the internal mechanism is switched to a retracting mode and for which subsequent pulls result in retracting the cutting implement. In a fourth embodiment, a surgical device comprises a pull lever 2502 and a toggle switch 2505. The pull lever may be used for actuating an impacting or advancement mechanism, and the toggle switch may be used to change the mode from advance to retract based on its position.

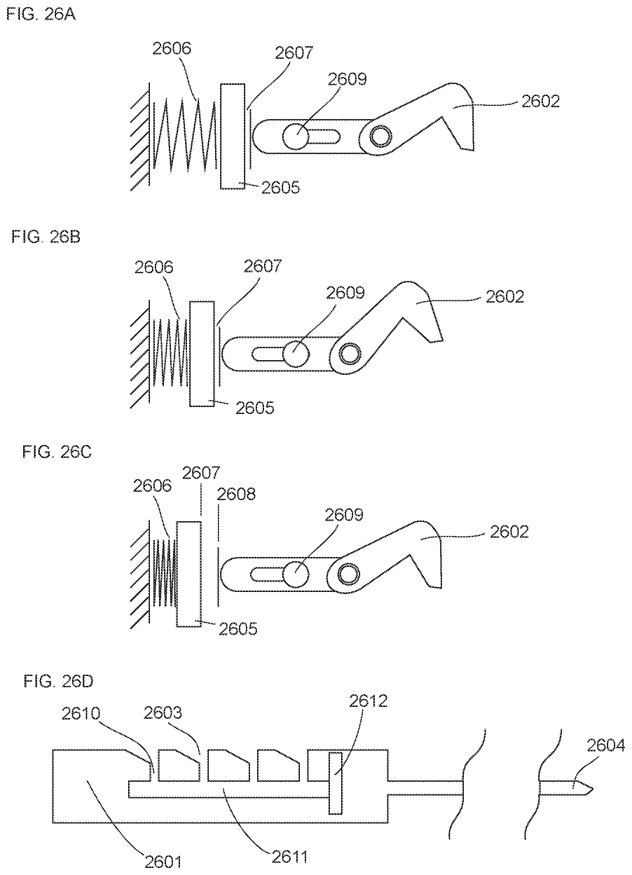

The system of FIG. 26A through 26D comprises a carriage 2601 with two or more indexed notches 2603 and a carriage driver 2602. The carriage is coupled to a cutting implement 2604 and advanced or retracted by the carriage driver, which is optionally set in motion via a biasing element 2606. In one embodiment, a contact surface 2607 on an impactor 2605 coupled to the biasing element impacts the carriage driver or a receiving surface 2608 of a carriage driver connected body or transmission. Each impact drives the carriage a predetermined distance on the index. The carriage driver initial and/or final position is optionally constrained by a limit stop 2609, which controls maximum travel and may enable the impactor contact surface to separate from the receiving surface, thereby creating a gap for acceleration and building momentum prior to impact. The indexed carriage optionally includes a track 2611 and a catch 2612 for retraction. When a user decides to retract the cutting implement, they may change the position of the carriage driver with respect to the carriage, for example and not limitation, by rotating the carriage and allowing the driver to slide along a channel 2610 until the driver reaches the track 2611, and then moving the driver distally to a catch 2612. With the driver releasably connected to the catch, the user may apply an actuator to retract the carriage, and then optionally move the driver back into index notch contact position for subsequent carriage advancement.

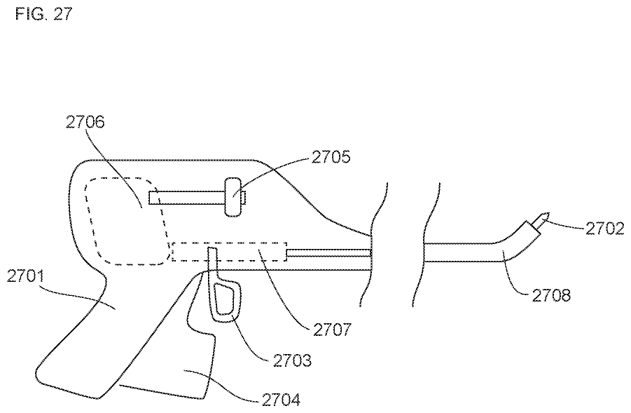

The surgical tool of FIG. 27 comprises a handle 2701, a hollow shaft 2708, a cutting implement 2702, and three actuators 2703 2704 2705. In a primary embodiment, a hollow shaft includes a bend. Further, the surgical tool depicted comprises an internal energy storage element 2706 and an advancement mechanism 2707. In one embodiment, a user applies a trigger actuator 2703 to drive the cutting implement forward for a high velocity impact; then a user may apply a lever actuator 2704 to controllably advance the cutting implement a known distance distally; and finally the user may utilize a sliding actuator 2705 to retract the cutting implement proximally from the patient site. In a second embodiment, sliding actuator 2705 is used to charge a biasing element; then trigger actuator 2703 is used to effect impact; then lever actuator 2704 is used to advance cutting implement; then trigger actuator 2703 is activated a second time to cutting implement direction of movement; and finally the lever actuator 2704 is used to retract the cutting implement. The functions of any of the aforementioned actuator types may be interchanged, and the forms of first, second, and third actuator may be different in an alternative embodiment.

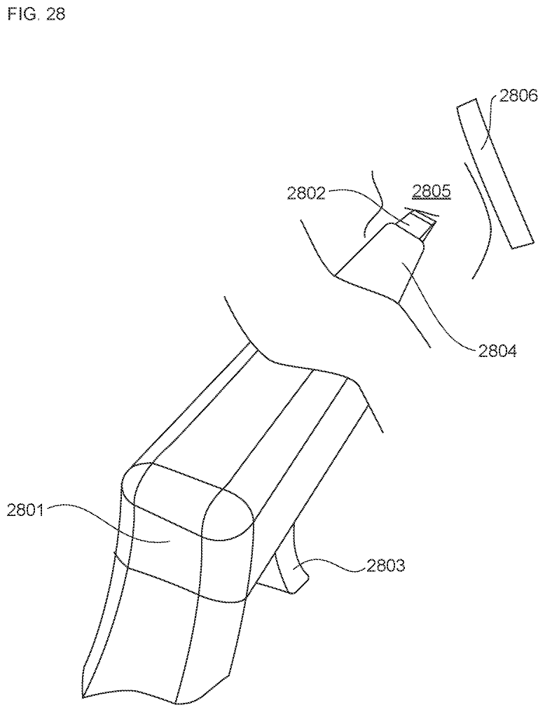

The surgical tool depicted in FIG. 28 represents a handheld mechanically assisted osteotome in use. The surgical tool of the present invention comprises a handle 2801, at least one actuator 2803, a hollow shaft 2804, and a cutting implement 2802. Upon actuating, a user may controllably transmit a predetermined force to the cutting implement, optionally limited to a fixed maximum travel distance. In mechanically constraining the force and/or travel of the cutting implement upon actuation, the user can more safely cut bone or target tissue 2805 while avoiding sensitive anatomy 2806. Further, relatively high velocity and low travel impact, advancement, oscillation, or vibration, reduces the likelihood of slipping or overshooting the target.

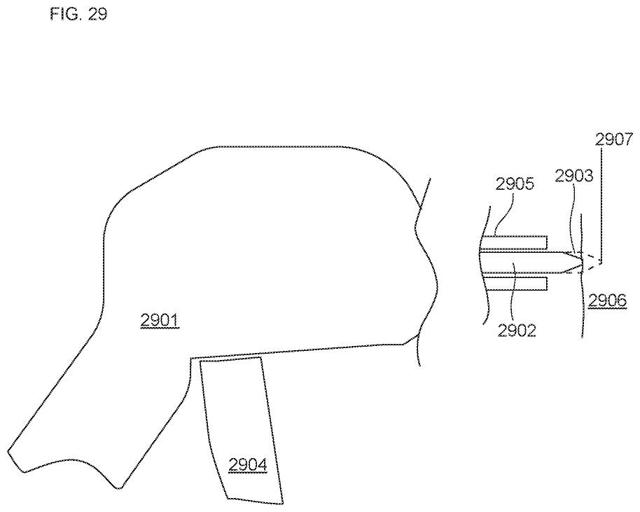

The surgical tool of FIG. 29 comprises a handle 2901, an actuator 2904, a hollow shaft 2905, and a cutting implement 2902. A user may utilize the actuator 2904 to effect impact or advancement of the cutting implement to a predictable final position 2903 and associated maximum depth 2907 with respect to the hollow shaft and into bone or tissue site of interest 2906, by means of consistent automated mechanical force delivery.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A surgical device for impacting and advancing an elongated body into and through a bone, the handheld surgical device including a handle, and a hollow shaft, said handle including an impacting mechanism and a manual advancing mechanism.

2. The surgical device of claim 1 further including a retracting mechanism.

3. The surgical device of claim 2 further including a retractor catch, a track and an index.

4. The surgical device of claim 2 further including a pull lever for charging the device and an actuation control for the impacting mechanism.

5. A method for advancing an elongated cutting body through bone using a surgical device, the method comprising;
a. Impacting a bone with an impacting mechanism to create an opening in the bone;
b. Advancing a elongated cutting body of the impacting mechanism with an advancing mechanism, into the opening in the bone a known distance; and
c. Retracting the elongated cutting body.
